(19) United States
(12) Patent Application Publication
(10) Pub. No.: US 2017/0231519 A1
(43) Pub. Date: Aug. 17, 2017

(54) SYSTEM AND METHOD FOR ANNOTATING AND ANALYZING EEG WAVEFORMS

(71) Applicants: The General Hospital Corporation, Boston, MA (US); NANYANG TECHNOLOGICAL UNIVERSITY, Singapore (SG)

(72) Inventors: Brandon Westover, Belmont, MA (US); Sydney S. Cash, Boston, MA (US); Justin Dauwels, Boston, MA (US); Jing JIN, Boston, MA (US)

(21) Appl. No.: 15/504,548

(22) PCT Filed: Aug. 19, 2015
(86) PCT No.: PCT/US15/45886

§ 371 Date: Feb. 16, 2017

Related U.S. Application Data
(60) Provisional application No. 62/039,070, filed on Aug. 19, 2014.

(51) Int. Cl. A61B 5/0478; A61B 5/00; A61B 5/04

(52) U.S. Cl. CPC A61B 5/0478; A61B 5/04017; A61B 5/742; A61B 5/6803

(57) ABSTRACT

Systems and methods of the present invention provide for storing an annotated set of confirmed epileptiform discharges (ED) waveforms in a database; receiving, by a computing device, a signal encoding electroencephalograph (EEG) data from a plurality of electrodes each attached to a subject and detecting EEG data; generating a user interface displaying a plurality of waveforms based upon at least a portion of the EEG data; receiving an initial selection of a portion of one of the plurality of waveforms comprising an ED; identifying a list of candidate waveforms including potential EDs by determining an alignment of the initial selection with a portion of one of the plurality of waveforms in the EEG data; displaying the list of candidate waveforms on the user interface; receiving, from the user and via the user interface, an identification of a subset of the list of candidate waveforms; and storing the subset of the list of candidate waveforms as an annotated list of confirmed EDs in the database.

### FIELD OF THE INVENTION

The present invention generally relates to the field of electroencephalography and specifically to a system and method enabling rapid waveform annotation used to generate a high volume database.

### SUMMARY OF THE INVENTION

The present invention provides systems and methods comprising: storing an annotated set of confirmed epileptiform discharges (ED) waveforms in a database; receiving, by a computing device, a signal encoding electroencephalograph (EEG) data from a plurality of electrodes each attached to a subject and detecting EEG data; generating a user interface displaying a plurality of waveforms based upon at least a portion of the EEG data; receiving an initial selection of a portion of one of the plurality of waveforms comprising an ED; identifying a list of candidate waveforms including potential EDs by determining an alignment of the initial selection with a portion of one of the plurality of waveforms in the EEG data; displaying the list of candidate waveforms on the user interface; receiving, from the user and via the user interface, an identification of a subset of the list of candidate waveforms; and storing the subset of the list of candidate waveforms as an annotated list of confirmed EDs in the database.

The above features and advantages of the present invention will be better understood from the following detailed description taken in conjunction with the accompanying drawings.

### BRIEF DESCRIPTION OF THE DRAWINGS

FIG. 1 illustrates a possible system for annotating and analyzing EED waveforms.

FIG. 2 shows a flow diagram illustrating a possible embodiment of a method for annotating and analyzing EEG waveforms.

FIG. 3 shows a flow diagram illustrating a possible embodiment of a method for annotating and analyzing EEG waveforms.

FIG. 4 shows a flow diagram illustrating a possible embodiment of a method for annotating and analyzing EEG waveforms.

FIG. 5 shows a flow diagram illustrating a possible embodiment of a method for annotating and analyzing EEG waveforms.

FIG. 6 shows a flow diagram illustrating a possible embodiment of a method for annotating and analyzing EEG waveforms.

FIG. 7 illustrates a comparison of a similarity search using a EuD (EuD) and a dynamic time warp (DTW) template matching algorithm.

FIG. 8 illustrates a EuD triangle inequality analysis.

FIG. 9 shows a graphical illustration of improvements to EuD analysis using online machine learning.

FIG. 10 shows a flow diagram illustrating a possible embodiment of a method for annotating and analyzing EEG waveforms.

FIG. 11 shows a flow diagram illustrating a possible embodiment of a method for annotating and analyzing EEG waveforms.

FIG. 12 illustrates a peak and trough of an EEG waveform.

FIG. 13 illustrates a user interface used in annotating and analyzing EEG waveforms.

FIG. 14 illustrates a user interface used in annotating and analyzing EEG waveforms.

FIG. 15 illustrates a user interface used in annotating and analyzing EEG waveforms.

FIG. 16 shows a flow diagram illustrating a possible embodiment of a method for annotating and analyzing EEG waveforms.

FIG. 17 shows a flow diagram illustrating a possible embodiment of a method for annotating and analyzing EEG waveforms.

FIG. 18 shows a flow diagram illustrating a possible embodiment of a method for annotating and analyzing EEG waveforms.

FIG. 19 shows a flow diagram illustrating a possible embodiment of a method for annotating and analyzing EEG waveforms.

### DETAILED DESCRIPTION

The present invention will now be discussed in detail with regard to the attached figures that were briefly described above. In the following description, numerous specific details are set forth illustrating the Applicant's best mode for practicing the invention and enabling one of ordinary skill in the art to make and use the invention. It will be obvious, however, to one skilled in the art that the present invention may be practiced without many of these specific details. In other instances, well-known machines, structures, and method steps have not been described in particular detail in order to avoid unnecessarily obscuring the present invention. Unless otherwise indicated, like parts and method steps are referred to with like reference numerals.

The capturing of Electroencephalogram (EEG) data involves recording of the brain's electrical activity from sensors placed on a subject's scalp; it is a measure of voltage fluctuations resulting from the ionic current flow within neurons of the brain, For a particular subject, EEG data can provide a continuous measure of cortical function with good temporal resolution. Consequently, EEG is often used in the diagnosis and management of neurological disorders such as epilepsy, sleep disorders, and encephalopathy, which can cause obvious abnormalities (i.e. special EEG waveforms) in EEG readings. For example, EEG recordings an important source of information about the neurological disorder known as epilepsy. EEG signals in people with epilepsy may contain waveforms known as epileptiform discharges (ED), also commonly referred to as “spikes” or “sharp waves,” which can be indicators of some abnormality or problem within the subject. Hereafter, we will use the terms ED and spikes interchangeably.

Epilepsy refers to a group of chronic brain disorders characterized by recurrent unprovoked seizures. EEG is the primary diagnostic test for epilepsy because EDs are a key diagnostic biomarker for epilepsy. When reviewing EEG data for a subject with epilepsy, for example, EDs show up within EEG signals as morphologically defined events that are paroxysmal, clearly distinguishable from the background with abrupt changes in polarity, and characterized by sharp contours. Because EDs occur almost exclusively in EEG data from epileptic patients, the presence of EDs predicts seizure recurrence, and facilitates the diagnosis of epilepsy, enabling appropriate treatment to be prescribed.

EEG interpretation is therefore conventionally performed by physicians with subspecialty training in neurology and clinical neurophysiology. This expertise requires many years of specialized medical training and exposure to 1000s of EEGs from a wide range of patients. When analyzing a patient's EEG data, physicians detect EDs by visually inspecting several EEG waveforms at a time. However, EDs within these various waveforms can be difficult to detect in a consistent manner due to wide patient variability and other factors. Additionally making the analysis of EEG data difficult, in many cases EEG data recordings can last from 30 minutes to several weeks, resulting in a vast amount of data to analyze.

Expert visual inspection and manual annotation are still the gold standard for interpreting EEG data. However, the process can be tedious and ultimately subjective—the agreement rate for the identification of EDs has been found as low as 60% between electroencephalographers for certain cases. In addition, EEGs are frequently misinterpreted by neurologists without specialized neurophysiology training, due to (i) the wide variety of morphologies of EDs and (ii) the similarity of EDs to wave shapes that are part of the normal background activity and to artifacts (e.g., potentials from muscle, eyes, and the heart) that are quite normal. As depicted in FIG. 2 (representing 200 EDs chosen at random from a database of 35,000 EDs of 303 different patients), there is a great variety in the morphology of the EDs.

Due to the difficulty of analyzing EEG data, many patients go undiagnosed and untreated, or are misdiagnosed by unqualified practitioners, leading to inappropriate medical interventions and avoidable suffering. Consequently, there is a need for an automated ED detection system. Such a system could potentially provide ED detection in a manner that is more efficient and reliable, and at lower cost, than is currently possible. Additionally, an automated system could potentially be widely deployed, overcoming the problem that qualified EEG experts are in short supply.

A hurdle to achieving a strong algorithm for ED detection is the lack of a sufficient database of annotated EEGs, which would provide a large number of exemplar EDs that may be referenced in identifying new EDs in new data. The primary challenge in obtaining such a database is that detailed manual annotation of waveforms can be slow and labor intensive, which can limit the potential sources of such annotated EEG data.

The brute-force approach to generating such a database is to manually annotate numerous EEG records. However, exhaustive manual annotation of EDs is prohibitively time-consuming, especially for EEG recordings with large numbers of EDs (e.g., up to thousands per hour). The time and labor required severely limits the availability of EEG experts to help establish a large database of annotated EDs.

Automated ED detection could be faster, less expensive, more objective, and potentially more accurate through the use of automated ED detection and classification schemes. Automated ED detection would enable wider availability of EEG diagnostics and more rapid referral to qualified physicians who can provide further medical investigation and interventions.

To address these issues, the present disclosure provides a system and method for at least partially automated EEG review and rapid waveform annotation. At the system's core lies a waveform analysis engine that performs template waveform matching using matching algorithms such as EuD and online machine learning and/or Dynamic Time Warping (DTW), which may substantially accelerate the task of annotating waveforms. These algorithms are described in more detail below.

The disclosed system and method are not limited to the annotation of EDs in scalp EEG recordings, but can also be readily generalized to other waveforms and signal types. The present disclosure, however, provides a number of examples involving the automated analysis and classification of waveforms derived from EEG data for evaluating patients for epilepsy, though it should be understood that the teachings of the present disclosure are equally applicable to other applications involving the analysis of waveforms describing EEG data for other purposes, and for other types of data. Examples of additional applications to EEG include but are not limited to the detection of waveforms characteristic of sleep (e.g. “spindles”, “K-complexes”, and “vertex waves”) and encephalopathy (e.g. “triphasic waves”). Examples of other medical data to which the invention is applicable include but are not limited to electrocardiogram (ECG) data (e.g. abnormal heart beat waveforms), respiratory time-series (e.g. abnormal breathing patterns such as apnea events), and imaging applications (e.g. annotation and detection of anatomical structures in MRI and CT images).

The present system includes a graphical user interface (GUI) designed for EEG review and rapid waveform annotation. To provide rapid annotation, in one embodiment, the system utilizes custom-built algorithms based on a combination of technologies of template matching and online machine learning. Once a user has selected an initial waveform such as an ED, that initial waveform is designated as a template. Template matching techniques can then be utilized to automatically generate a list of waveform candidates that may each contain an ED having a similar shape to that selected by the user. Once a set of waveform candidates is identified, the candidates are displayed back to the user, who can confirm the candidates that do, in fact, contain EDs. Online machine learning can then be used following this initial template matching step to generate a refined set of waveforms, each containing potential EDs. An alternative embodiment relies upon an approach employing DTW-based template matching to identify potential EDs.

The ED-matching approaches underlying the present system are based on the observation that, within the same patient or subject, EDs typically share a similar morphology. Thus, within particular subjects, waveforms such as EDs tend to be fairly stereotyped. Consequently, the identification of one example ED waveform as a template waveform for a particular subject can be utilized to enable rapid and automatic identification and extraction of many more candidate matching waveforms from the subject's EEG data, which can then be further accepted or rejected by an EEG reviewer.

With a suitable choice of similarity measure and ED templates, it therefore may be possible to extract many more similar candidate waveforms from the same EEG record in less time. Rather than annotating one ED at a time, for example, groups of potentially-similar EDs (typically 10-100 of EDs) can be identified and annotated by template matching, accelerating the review and annotation process.

The present system may also employ a cascade of differentiated classifiers for progressive background rejection in order to overcome the between-patient variability of EDs to achieve expert-level automated ED detection. Thus, the disclosed invention further comprises a fully automated ED detection algorithm that performs analogous to a human expert.

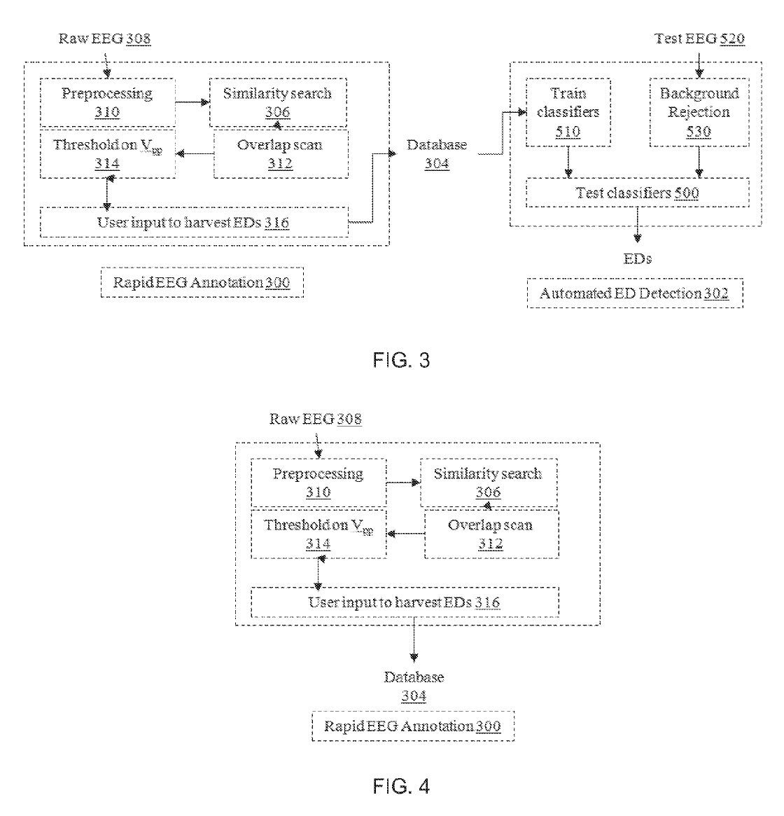

FIG. 3 is a flowchart demonstrating an overview of the disclosed system, which comprises two general steps involving rapid EEG annotation and automated ED detection. FIG. 4 is a flowchart demonstrating further detail of the first general step of rapid EEG annotation 300, and FIG. 4 is a flowchart demonstrating further detail of the second general step of automated ED detection 302. As seen in FIG. 3, the user input to harvest EDs generated from rapid EEG annotation 300 may be stored in a database 304 used to train classifiers for automated ED detection 202.

FIG. 4 shows sub-steps of the first general step seen in FIG. 3, and depicts an algorithm for EEG review and template matching enabling rapid EEG annotation 300, thereby providing doctors and technicians with the ability to quickly template and annotate a plurality of EDs and populate a database 304 for algorithmic learning purposes. This rapid EEG annotation algorithm 304 can be accomplished through an interactive process wherein a user selects or identifies a particular ED contained within the EEG data for a subject. That identified ED then becomes a template and the system employs a similarity search algorithm 306 (e.g., DTW or EuD/online machine learning, described below) to identify a number of waveforms within the subject's EEG data that match the template. These waveforms are displayed as a list or cluster to the user, who can then select and verify that the waveforms recognized by the template matching algorithm do in fact depict EDs.

Before the EEG data can be analyzed according to the present methods, the EEG data may be pre-processed. FIG. 6 is a flowchart representing a more detailed view of the pre-processing step (Step 310) seen in FIGS. 3 and 4. The method initially receives raw EEG data 308. To make this raw EEG data 308 more well-suited to analysis, and thereby improve the efficiency and accuracy of the disclosed system, the sampling rate of the raw EEG data may be reduced (Step 600), the raw EEG data 308 may also be subjected to various filters (Step 610), or other modifications as part of preprocessing. In one embodiment, preprocessing may involve down-sampling the raw EEG data 308 to 128 Hz in order to reduce computational complexity (visual recognition of EDs in scalp EEG data may be uncompromised by sub-sampling to this level). Digital filters such as a 60 Hz notch filter and [0.1 Hz 64 Hz] band-pass filter can also be applied to remove artifacts such as power line interference and baseline fluctuations.

The preprocessing (Step 310) of the raw EEG file 308, described in the preceding paragraph, may produce clean EEG data 620 used to perform template matching (Step 710, below) within the similarity search 306.

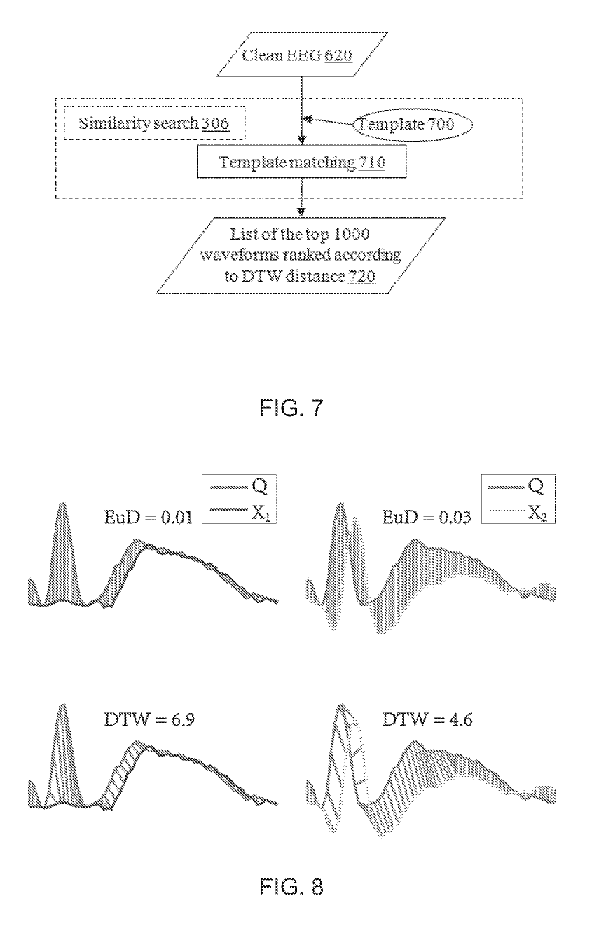

After the raw EEG data has been pre-processed (Step 310) as illustrated in FIG. 6, template matching (Step 710) can be performed on the clean EEG data 620 to identify waveforms that match a template initially selected by a user. FIG. 7 is a flowchart representing a more detailed view of the steps involved in the user identifying a template 700 and then the system executing template matching (Step 710). After the raw EEG data 308 has been preprocessed (Step 310), the clean EEG data 620 can be presented to the user, for example, via the user interface shown in FIG. 14 and described below. Using the user interface, the user may provide a template ED 700 (e.g., by selecting or otherwise identifying an ED that appears in the EEG data depicted within the user interface), and the disclosed system runs a template marching algorithm 710 to find template-matching EDs within the clean EEG data 620.

In selected embodiments, the template matching algorithm used to execute step 710 of the similarity search can include a EuD algorithm or a DTW algorithm, the use of both being novel and interchangeable with the disclosed system. The EuD algorithm is based on a simple one-to-one alignment of waveforms that can be computed rapidly. This approach may include a high sensitivity to small variations in the morphology of the waveforms, which does not emphasize morphological features of an ED such as the sharp contour.

In some cases, the use of a DTW-based template-matching algorithm 710 may improve upon a EuD-based approach. As seen in FIG. 8, DTW (see the waveforms of FIG. 7b) permits non-linear distortion of the time axis to achieve better waveform alignments as compared to EuD approaches (see the waveforms of FIG. 7a). In DTW, therefore, segments of a time series are aligned with segments of another time series, effectively allowing for matching similar waveforms in spite of small local dilations and contractions of the time axis. In the standard implementation of the DTW algorithm, optimal alignment of waveforms is accomplished through an iterative process known as dynamic programming algorithm. In effect, DTW allows for small localized stretching and dilation (“warping”) of the time axis to minimize the distance between a pair of morphologically-similar waveforms, where “distance” is measured by the EuD of the warped waveforms. Fast versions of the DTW algorithm have also been recently discovered, and may be used for template matching in the disclosed invention. Further details of the DTW algorithm and its fast implementation are provided below.

Using EuD algorithms to perform template matching may include generating a list of waveform candidates based on the z-normalized EuD (although other measures could be used as well) computed with respect to the template waveform selected by the user.

When performing EuD matching, the EuD between 2 time series Q=q₁; q₂; . . . qₙ and X=x₁; x₂; . . . ; xₙ of the same length is defined as:

ED(Q,X)=\sqrt{Σᵢ₌₁ⁿ(q₁−x₁)ⁿ}

The EuD is based on a simple one-to-one alignment of waveforms that can be computed rapidly. In a EuD based approach, template matching may be carried out based on the z-normalized EuD.

In the context of the current invention, the EuD approach may be used to measure the similarity between the template ED 700 provided by the user and any matches to the template (Step 710) found within in the clean EEG data 620. For each record, a distance lookup table may be computed beforehand with respect to the same randomly selected reference. To reduce computational complexity, the triangle inequality may be applied to reject samples far away from the given template, and narrow down the range of search to a small group of samples. The accepted samples may be further ranked according to the EuD to the given template in ascending order.

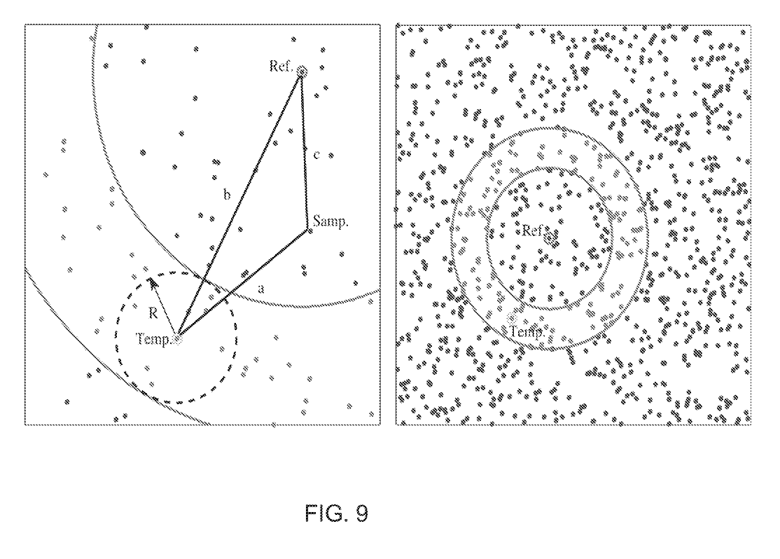

For each EEG recording, a distance look-up table (LUT) is computed beforehand with respect to a reference randomly selected. To reduce the computational complexity, triangle inequality is used with the LUT to abandon waveforms that are far away from a given template. In FIG. 9, each waveform can be represented by a single point in the domain of distance. For each template, a triangle is formed by 3 points: the template (Temp.), the reference waveform (Ref.) used for LUT computation, and a third point (Samp.) representing any sample waveform in the EEG, with (a, b, c) denoting the lengths of the sides of the triangle. A sample waveform (Samp.) is abandoned if |b−c|>R, with R denoting the tolerance of similarity search. As a result, the searching range is narrowed to a much smaller group of samples bounded by 2 circles defined by R. The remaining sample waveforms are further ranked according to the Euclidean distance to the given template in ascending order.

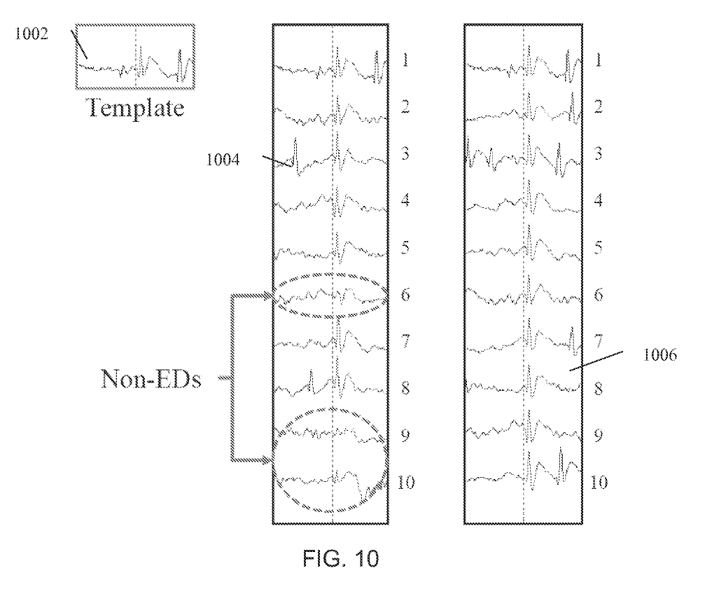

Although fast, using EuD as the method of template matching has its drawbacks, resulting in occasional bad rankings in the list of waveforms. With feedback from the user (e.g., waveform selections subsequent to the first selected waveforms), the annotation may be cast into an online machine learning task. By continuously learning from previous annotations, the current ranking in the list may be refined by applying online machine learning. FIG. 10, for example, depicts a first set of candidate ED waveforms 1004 that may have been identified using EuD for template matching based upon template waveform 1002. Following the application of online machine learning, however, a number of the candidate waveforms may be identified as non-ED waveforms and revise setup of ED-containing waveforms 1006 can be generated.

As described below, following the template matching step in the similarity search, a user may select or identify waveforms identified by template matching that do, in fact, match the user's selected template. With feedback from the user, the annotation (e.g., the indication of whether the waveforms identified by template matching matched the user's template) can be cast into an online machine learning task. By continuously learning from the user's previous annotations, the current ranking in the list can be refined by applying online machine learning. Online machine learning can be used afterwards to refine the ranking in the list for further selection.

Online machine learning is a model of induction that learns sequentially. A key defining characteristic of online machine learning is that the true label of the instance is revealed soon after the prediction is made, to refine the prediction hypothesis for future trials. Due to continual feedback from the user confirming whether identified waveforms actually contain EDs, the online learning algorithms are able to adapt and learn in difficult situations.

The goal of the online machine learning algorithm is to minimize some performance criteria which are algorithm specific. As a non-limiting example in the disclosed system, the MATLAB-based toolbox LIBOL may be applied to provide a collection of various online machine learning algorithms.

One aim of the disclosed invention is to achieve faster annotation of EEG data by a variety of strategies, including preprocessing (Step 310, described above), DTW, and clustering of EDs. As noted above, DTW permits non-linear distortion of the time axis to achieve better waveform alignments and provides an alternative to EuD algorithms for template matching. In DTW, segments of a time series (e.g., the template ED) are aligned with segments of another time series (e.g., potential matching EDs within the EEG data 520), effectively allowing for matching similar waveforms in spite of small local dilations and stretches of the time axis.

Template matching to perform the similarity search (Step 306) using the DTW algorithm may include aligning 2 time series Q=q₁, q₂, . . . , qₙ and X=x₁, x₂, . . . , xₙ, a warping matrix DεRⁿ^{^{z}} is constructed whose entries D_{i,f} are the following: D_{i,f}(Q,X)=|qᵢ−x_{f}|+min(D_{i-1,f}, D_{i-1,f-1}, D_{i,f-1}) The optimal warping path is obtained within the region constrained by the Sakoe-Chiba Band with a width of R (typically set to 10% of the signal's length).

Although DTW often yields good matches, it can be computationally expensive. Returning to FIG. 4, the efficiencies of the DTW algorithm may be improved by performing pre-processing (Step 310—shown and discussed above in greater detail in relation to FIGS. 3, 4 and 6), an overlap scan (Step 312—shown in greater detail in FIGS. 3, 4 and 11), an assessment of threshold on Vₚₚ (Step 314—shown in greater detail in FIGS. 3, 4 and 17), and clustering EDs during the user's assessment of candidate matching EDs in order to harvest EDs (Step 316—shown and discussed in greater detail in FIGS. 3, 4 and 16).

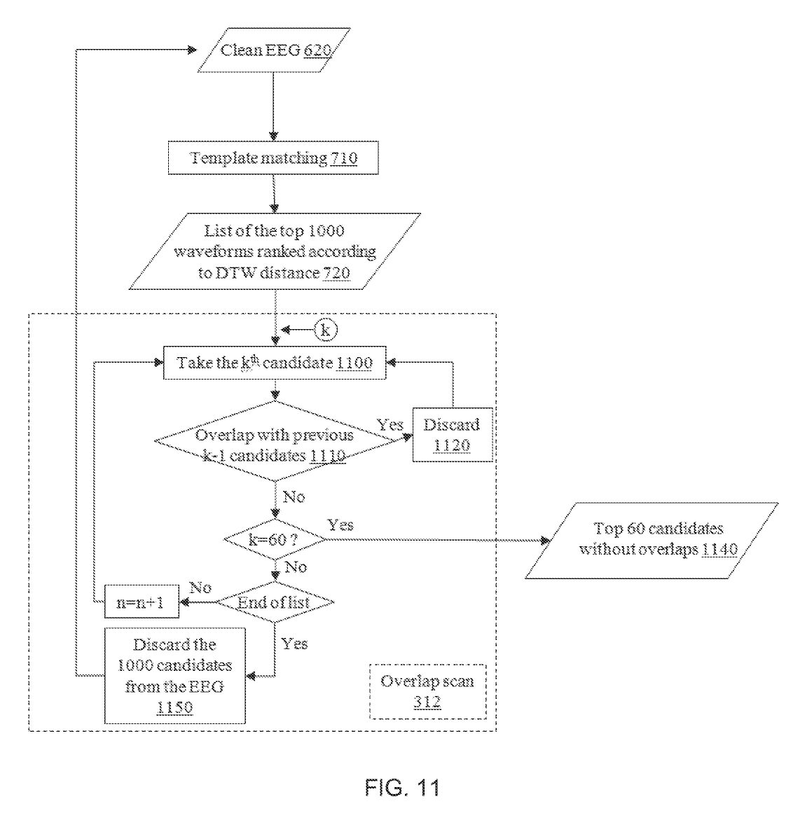

FIG. 11 is a flowchart representing a more detailed view of the overlap scan seen in FIGS. 3 and 4. This overlap scan may be used to remove overlaps from the ED waveforms within the EEG data matching the template provided by the user. Overlap of these ED waveforms may be defined as one or more waveforms in the EEG data from the list of candidate waveforms wherein a portion of a waveform in the EEG data overlaps a portion of one of the candidate waveforms in the list of candidate waveforms. As seen in FIG. 7, and described in greater detail below, the output of the template matching algorithm (Step 710), may comprise a list of the top 1000 ED waveforms within the EEG data matching the template, ranked according to the DTW value relative to the template 700.

Within this output of matching waveforms, there are typically numerous candidates with some degree of overlap. That is, waveform candidates that include overlapping data points, or data points that occur at the same time or within a threshold time period of one another. In some embodiments, the template matching algorithm may apply a sliding window when extracting potential ED waveform candidates matching the template provided by the user. The sliding window has a length of n and moves 1 data point each time along the time series X=x₁, x₂, . . . , xₙ with n being the length of the template, and N the length of the input EEG. For instance, if the candidate waveform Xₘ=xₘ, xₘ₊₁, . . . , xₘ₊ₙ₋₁, has a high ranking in the list, finding X_{m±1} also in the list with similar rankings is likely (Steps 1100-1110). To remove candidates with large overlaps, the list of waveforms is scanned from top to bottom. Each candidate found to have more half of the window length overlapping with any candidate possessing a higher ranking is discarded from the list (Step 1120).

The overlap scan stops when there are 60 candidates 1140 with less than half of the window length of overlap (Step 1130). If there are less than 60 candidates after scanning the list of all 1,000 waveforms, the disclosed system discards these 1,000 waveforms (Step 1150) from the input EEG (Step 1120). The disclosed system then selects the next 1,000 DTW-based candidates 620 and repeats the overlap scan to identify more candidates (Steps 1100-1110). Ultimately, this process yields at most 60 candidates with less than half of the window length of overlap 1040, ready to be passed to the user for assessment. As seen below, the remaining candidates may be grouped into dusters of 10 waveforms each, according to the characteristic Vₚₚ.

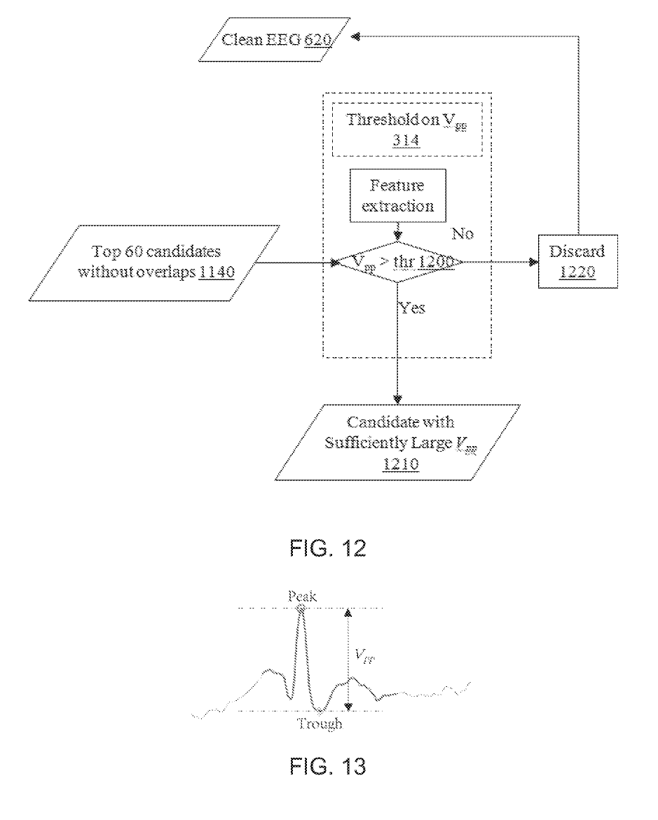

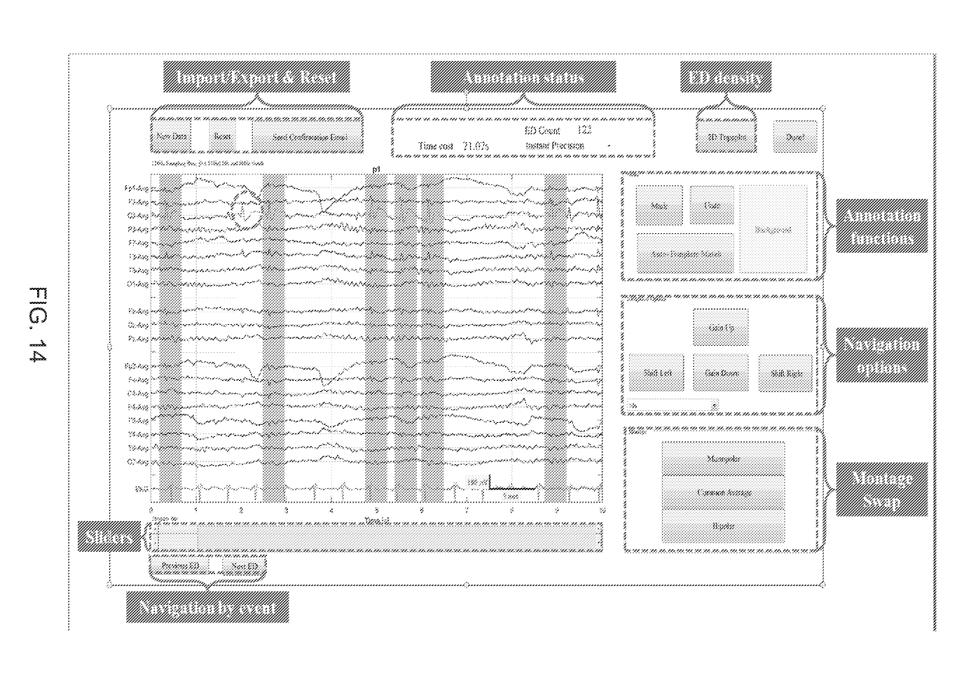

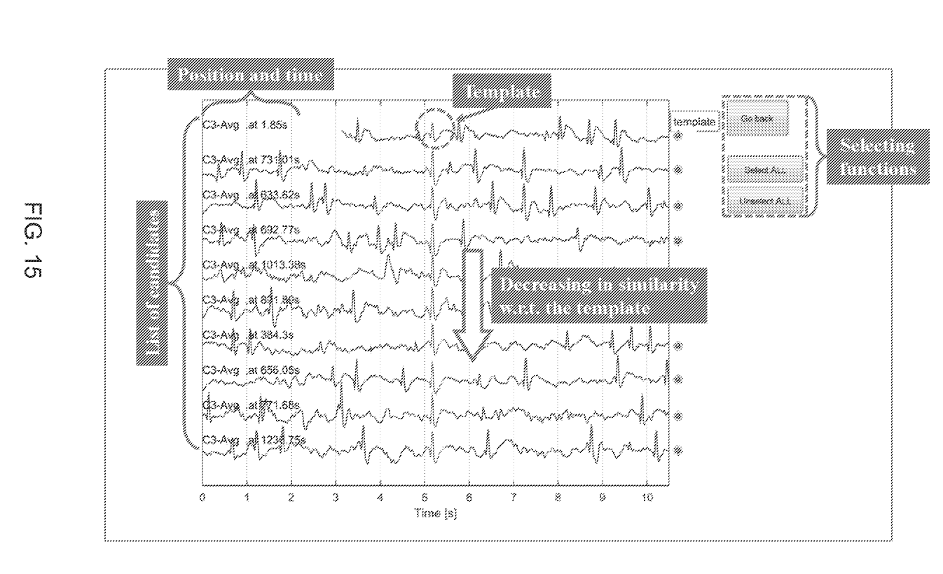

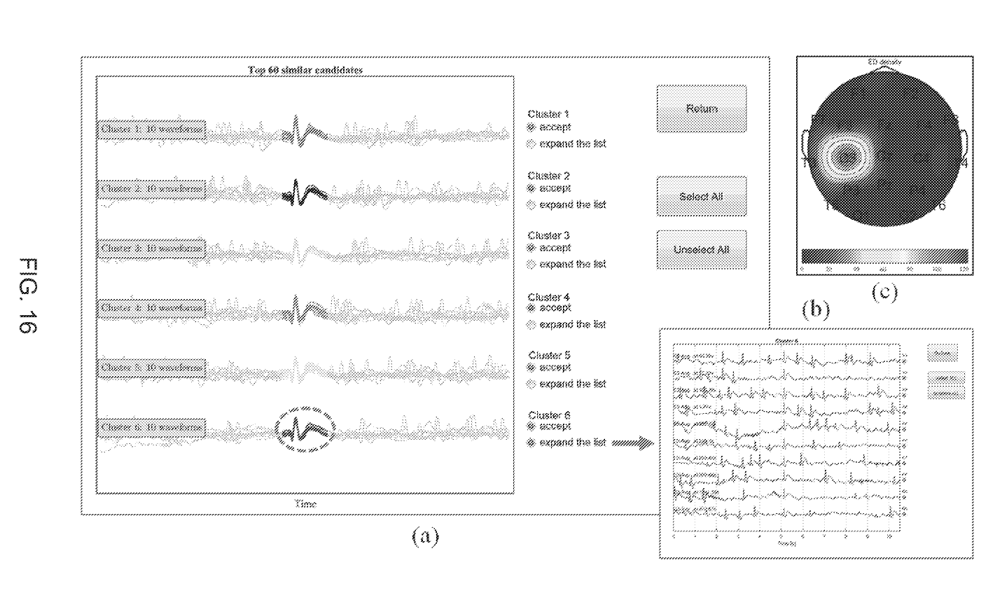

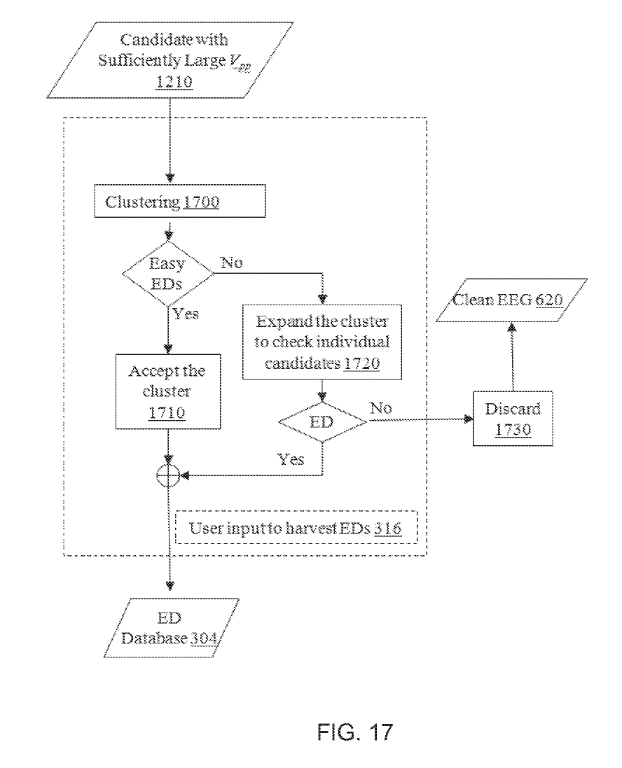

FIG. 12 is a flowchart representing a more detailed view of the assessment of threshold on Vₚₚ (Step 314) seen in FIGS. 3 and 4. The disclosed invention utilizes z-normalization, and as it does so, low amplitude spike candidates may be enlarged to appear as legitimate ED candidate and may be, consequently, detected. However, these low amplitude EDs are often determined to be false detections by EEG experts. In the context of FIG. 17, the top 60 candidates without overlaps 1140, generated from the overlap scan process in FIG. 11, is received. In order to eliminate low amplitude false detections, the peak-to-trough value Vₚₚ is extracted from each of the top 60 candidates (Step 1200), FIG. 13 demonstrates an example peak to trough value Vₚₚ.

A threshold is applied to each for the 60 candidate wave forms, such that only candidates with Vₚₚ, greater than the threshold 1210 are kept, which takes the value of 95% of the minimum peak-to-trough voltage (Vₚₚ)ₘᵢₙ obtained from existing annotated EDs. Candidates with Vₚₚ, less than the threshold are discarded (Step 1220) and the Clean EEG is modified accordingly.

In some embodiments, the efficiency of the DTW algorithm may be further improved through use of a modification of the Trillion algorithm from the UCR (University of California, Riverside) suite. The disclosed invention utilizes the most recently annotated ED as the template, and executes the Trillion algorithm for rapid DTW searching for template matching. The UCR suite draws on four ideas to reduce the computational complexity and increase the speed of DTW, namely: early abandoning z-Normalization; reordering early abandoning; reversing the query/data role in lower bound computation; and cascading lower bounds. As a result, DTW can be applied to relatively large datasets, including EEG recordings used for ED detection.

One technique used in the Trillion algorithm to speed up template matching with an expensive distance measure such as DTW is to use a cheap-to-compute lower bound to prune off unpromising candidates, in order to interleave early abandoning calculations of the lower bound with online z-normalization to optimize the normalization step. In other words, as the z-normalization is incrementally computed, the disclosed system may also incrementally compute the lower bound of the same data point. Thus, if this computation can be abandoned early, not only can the distance calculation be pruned, but also the normalization steps.

In similarity search, each subsequence needs to be normalized first. In the Trillion algorithm, the mean of the subsequence can be obtained by keeping two running sums of the long time series, which have a lag of exactly m values. The sum of squares of the subsequence can be similarly computed. The formulas are given below for clarity:

$$μ = \frac{1}{m} ( {\sum }_{i = 1}^{k} {x}_{i} - {\sum }_{i = 1}^{k - m} {x}_{i} ) , {σ}^{2} = \frac{1}{m} ( {\sum }_{i = 1}^{k} {x}_{i}^{2} - {\sum }_{i = 1}^{k - m} {x}_{i}^{2} ) - {μ}^{2} .$$

Online normalization in the Trillion algorithm enables early abandoning of the distance computation of the lower bound in addition to the normalization. A high-level outline of the algorithm shown in Table 1 below.

TABLE 1
Subsequence search with online z-normalization
(Rakthanmanon et al., 2012).

| Algorithm Similarity Search | |
|---|---|
| Procedure [nn] = SimilaritySearch (T,Q) | |
| 1 | best-so-far←∞, count←0 |
| 2 | Q←z-normalize(Q) |
| 3 | while !next(T) |

| | |
|---|---|
| 4 | i←mod(count,m) |
| 5 | X[i]←next(T) |
| 6 | ex←ex+X[i], ex2←ex2+X[i]² |
| 7 | if count≧m−1 |

| | |
|---|---|
| 8 | μ←ex/m, σ←sqrt(ex2/m−μ²) |
| 9 | j←0, dist←0 |
| 10 | while j<m and dist<best-so-far |

| | |
|---|---|
| 11 | dist←dist+(Q[j]−(X[mod(i+1+j,m)]−μ)/σ)² |
| 12 | j←j+1 |

| | |
|---|---|
| 13 | if dist<best-so-far |

| | |
|---|---|
| 14 | best-so-far←dist, nn←count |

| | |
|---|---|
| 15 | ex←ex−X[mod(i+1,m)] |
| 16 | ex2←ex2−X[mod(i+1,m)]² |

| | |
|---|---|
| 17 | count←count+1 |

Instead of the conventional left-to-right ordering to incrementally compute the distance, the disclosed invention when using the Trillion algorithm for DTW calculations may utilize a universal optimal ordering. It is conjectured that the universal optimal ordering is to sort the indices based on the absolute values of the z-normalized Q. The intuition behind this idea is that the value at Qᵢ will be compared to many Xᵢ values during a search. However, for subsequence search with z-normalized candidates, the distribution of many Xᵢ values will be approximately Gaussian, with a mean of zero. Thus, sections of the query that are farthest from the zero mean will tend to have the largest contributions to the distance measure. This universal optimal ordering used in the Trillion algorithm has been empirically validated by comparing it with the empirically determined optimal ordering in a series of numerical experiments, yielding a correlation value of 0.999.

Usually, lower bounds are applied to build an envelope around the query Q. However, as discussed in the next section, envelopes can also be formed around the candidate X in a “just-in-time,” fashion, to handle the scenario where all other bounds fail to further prune the candidate waveform. In the Trillion algorithm, this removes the space overhead and the time overhead pays for itself by pruning more full DTW calculations.

An efficient strategy used in the Trillion algorithm to speed up time series similarity search is to use lower bounds to admissibly prune off unpromising candidates. The Trillion algorithm applies all of these lower bounds in cascade. The algorithm first considers the O(1) lower bound LB_{Kim}FL, which can be a weak but fast-to-compute lower bound that prunes many candidates. If a candidate is not pruned at this stage, the O(n) lower bound LB_{Keogh}EQ is considered. If this lower bound is completed without exceeding the best-so-far, the Trillion algorithm reverses the query/data role and computes lower bound LB_{Keogh}EC. If this bound does not allow pruning, the Trillion algorithm starts the early abandoning calculation of DTW.

Returning to FIG. 7, and as previously noted, the user may view the clean EEG 520 in order to select a template 700 for template matching (Step 710). To accommodate these steps, the user interface seen in FIG. 14 comprises a fully featured EEG viewer for EEG review and manual annotation. The clean EEG waveform is displayed with existing markers, if any. Each of the labels on the left hand side is a name of an electrode and a position on the head.

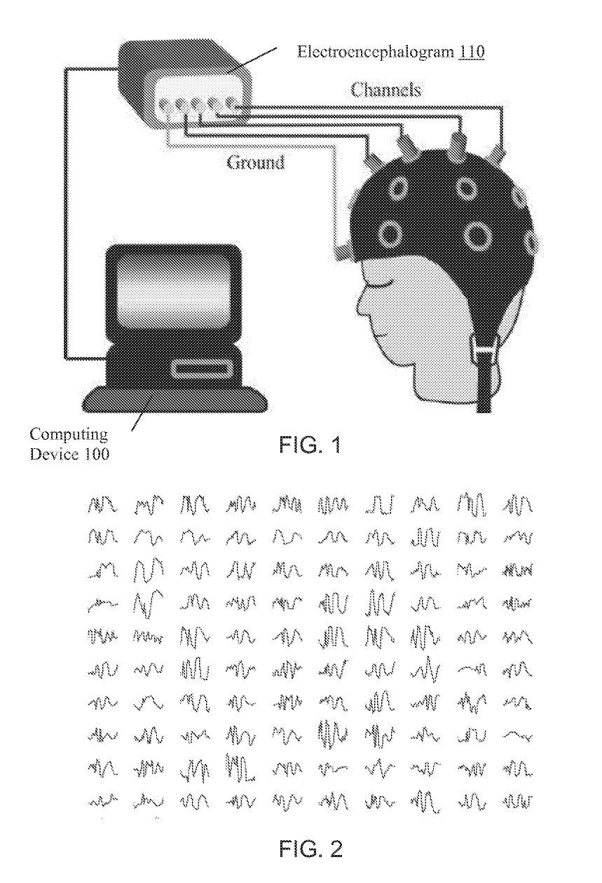

Returning to FIG. 1, the user interface may be displayed on a computing device such as a client or server 100 and may be any graphical, textual, scanned and/or auditory information a computer program presents to the user, and the control sequences such as keystrokes, movements of the computer mouse, selections with a touch screen, scanned information etc. used to control the program. The commands received, or any other information, may be accepted using any field, widget and/or control used in such interfaces, including but not limited to a text-box, text field, button, hyper-link, list, drop-down list, check-box, radio button, data grid, icon, graphical image, embedded link, etc.

The computing device may be any computer or program that provides services to other computers, programs, or users either in the same computer or over a computer network. Such computing devices may include, as non-limiting examples, a desktop computer, a laptop computer, a server computer etc.

The computing device may be communicatively coupled to data storage including any information requested or required by the system and/or described herein. The data storage may be any computer components, devices, and/or recording media that may retain digital data used for computing for some interval of time.

The user interface shown in FIG. 14 allows a user to display and navigate through EEG recordings along the time axis and to vary the display window duration. The user may navigate forward and backward to time points within the displayed clean EEG 620 at fixed steps (e.g., 5-10 seconds with or without interframe overlap respectively) or may apply the time slider at the bottom of the GUI to swiftly-shift to any time point.

The EEG viewer may comprise basic navigation functions such as shifting along time either at different step size or via a swift slider, amplitude scaling up/down, montage swap, and manual annotation. Montage swap buttons enable easy switching among the three most commonly used EEG data display schemes, i.e., mono-polar, common-average, and bipolar montage, catering to the needs of neurophysiologists to display EEG in different formats.

Apart from traditional navigation along the time axis with fixed time step-size or sliders, the disclosed system allows for navigation through the waveforms by annotation. EDs annotated can be reviewed or jumped to in the EEG data using the buttons “previous spike” and “next spike”, which jumps directly to the nearest (±1) waveform marker found in the record. Annotation status in terms of total current waveform count and online machine learning classification rate are shown at the top for the purpose of supervision.

The button “Auto-Template Match” activates the similarity search process using the template matching algorithms (i.e., EuD or DTW) disclosed above for rapid waveform annotation. To execute this function, the user may manually select a waveform template by left clicking the mouse at the waveform (right clicking to un-select) before pressing the button. Pressing the button triggers a similarity search.

FIG. 15 shows a list of candidate waveforms generated from the template matching (Step 710) performed as part of the similarity search 306 in response to the user selecting a template 700 and clicking the “Auto-Template Match” button. The displayed candidate waveforms matching the template may be ranked according to the similarity to the selected template in descending order. Newly-selected waveforms may be annotated automatically in the navigation window.

Thus, the disclosed system is semi-automated in the sense that users are actively involved in 2 types of tasks: (i) the user needs to provide templates 700 for EuD and/or DTW-based template matching algorithms, as seen in FIG. 14; and (ii) the user needs to provide an assessment of the displayed candidate waveforms matching the template (accepting or rejecting the suggested matches) to harvest EDs, as seen in FIGS. 2-3. More specifically, the user is required to determine whether the suggested waveforms are indeed EDs. In FIG. 15, selection or deletion of waveforms can be done by checking radio buttons within the list of candidate waveforms. The waveforms are ranked in a descending order of similarity to the template.

In addition to selecting individual candidate waveforms matching the template, as seen in FIG. 15, a user may select a duster of candidate waveforms, as seen in FIG. 16. The dusters of waveforms may be identified using any of the template matching and similarity search algorithms described herein. The user can assess the waveforms cluster-by-duster at a glance, and potentially accept an entire duster as confirmed EDs. The waveforms in each duster are overlaid, since these waveforms are usually very similar. If the user wishes to see the waveforms in a duster in more detail, the disclosed invention allows the user to expand a duster as shown in FIG. 16(b), providing a listing of the individual waveforms with detailed information to support the assessments, including both temporal and spatial coordinates, and 10s of context EEG. Another module displays the ED density on a topographic map (see FIG. 15(c)), showing the brain regions that exhibit EDs.

FIG. 17 is a flowchart illustrating a process of depicting a cluster of waveforms potentially including EDs to a user and receiving from the user an indication of whether the cluster, or individual waveforms in the cluster, do, in fact, depict EDs. The process may receive a candidate waveform with peak-to-trough value Vₚₚ, 1210 as previously seen in FIG. 12. In the example embodiment seen in FIGS. 16 and 17, the candidates are sorted into 6 clusters, according to the peak-to-trough value Vₚₚ, with 10 waveforms per cluster (Step 1700). The user can then assess the waveforms in the different clusters. The user is given the option to accept an entire duster at once as true ED waveforms (Step 1710), expand the duster to check individual candidates (Step 1720) or, if some of the candidates in a duster are not considered true EDs, the user can discard them (Step 1730).

In both EuD and DTW, once all candidates are returned and displayed to the user, the user may select all or just some of the candidates that they confirm are in fact representatives of the candidate signal of interest, or deselect those that are not. The end result is therefore that all EDs are certified by an expert's recognition as being valid. The process may then repeat until the user is satisfied that all the samples in a given EEG have been found and marked. This process may also be repeated for hundreds of EEGs, which are then moved into the database.

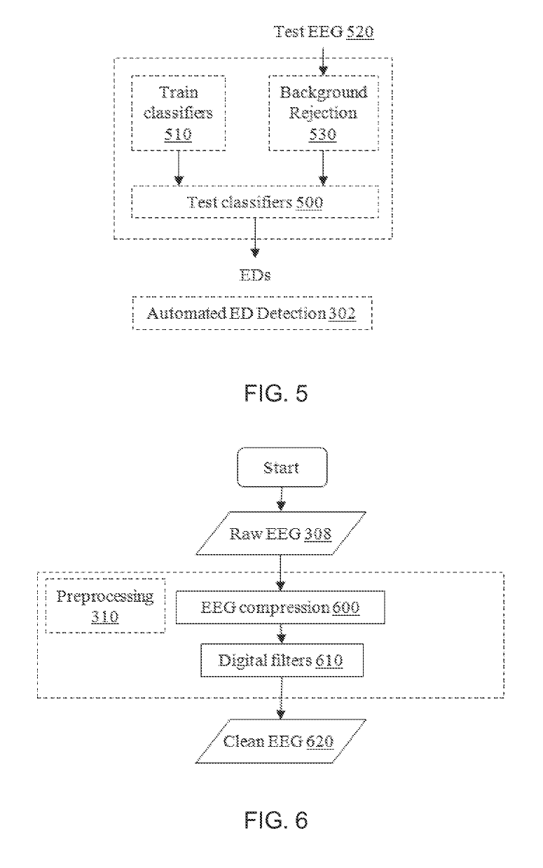

Returning now to FIG. 3, the confirmed and annotated EDs (Step 316) may be used in automated ED detection 202. This (preferably large) set of confirmed and annotated ED waveforms may be leveraged to develop a general purpose automated ED detection algorithm 202, seen in FIG. 5, representing the right side of the overall algorithm seen in FIG. 3. In FIG. 5, the classifiers to be tested against EEG data may be trained (Step 510) using the confirmed and annotated ED waveforms. A rest EEG 520 may also be used to determine background rejection 530 of data that is determined not to be EDs. Using the trained classifiers, the classifiers may be rested against incoming EEG data 520 in order to identify EDs.

To cope with the wide between-patient variability inherent in EEG data, one approach to classifier development may be based on the concept of classifier ensembles and cascades, i.e. building up a sophisticated classifier out of many simpler classifiers. Classifier cascades have the advantage of being able to deal with extreme pattern variability (no single classifier in the cascade is expected to do all the work), and computational efficiency.

Deep machine learning techniques may also be used to train (Step 510) and test (Step 500) the classifiers against EEG data 520. Employing a cascade of differentiated classifiers for progressive background rejection may overcome the between-patient variability of EDs to achieve expert-level ED detection. This method is similar to boosting, where a strong algorithm emerges from an ensemble of weak classifiers. Background waveforms are rejected partially at each step while reserving all or nearly all valid EDs.

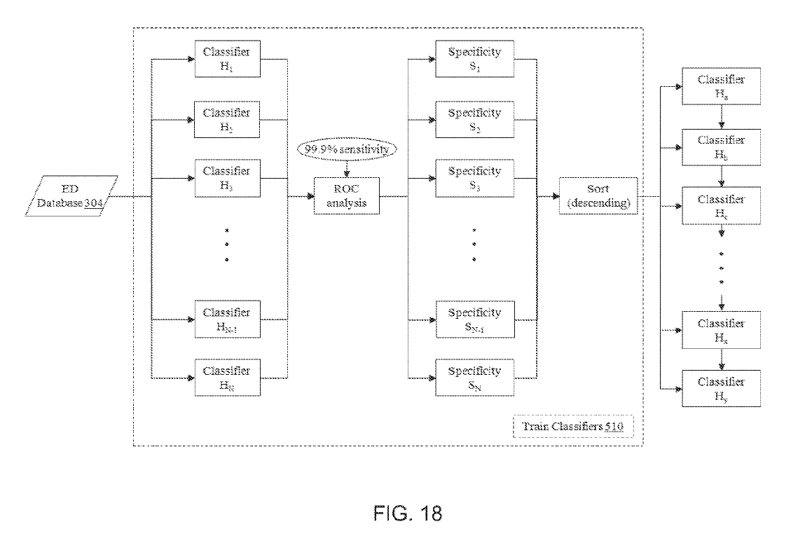

Returning now to FIG. 17, the confirmed and annotated EDs may produce a database of ED profiles 304. As seen in FIG. 18, the database of ED profiles 304 may be used to train a group of classifiers within the database. Each of these profiles may include labels, identifying each of the EEG waveforms as an ED or not. Each of the profiles may also include one or more features that define the ED. A large family of generated classifiers and/or potential classifiers may be evaluated and trained to determine ranks among the classifiers.

Simple classifiers (e.g., generated using extreme learning machine (ELM), support vector machine (SVM), or support vector regression (SVR) methods) are trained using the created database in order to generate a large pool of weak classifiers. Training of the overall ED detection system may include subjecting the EEG to a cascade of simple classifiers, beginning with classifiers that are simple filters that are designed to remove obvious background wave forms, then using progressively more complicated classifiers that may use more features or more intensive computations.

Training of classifiers may occur in a series, beginning with a simple classifier. As a simpler classifier makes mistakes, the incorrectly specified, or otherwise incorrect data may be used to train a second, more complex classifier, which will also make mistakes. These mistakes may be used to train a third, more complex classifier, and so on. Thus, the training scheme for the overall detection system may determine an order to rank how effective the classifiers are. Ranks are assigned using receiver operating characteristic (ROC) curves, derived by changing the discriminant threshold upon classification scores.

The threshold may be determined as the value for the threshold that preserves 99.9% of EDs (sensitivity). The rate of background rejected (specificity) may also be recorded. The classifiers can be sorted according to the specificity values in a descending order, to form a cascaded queue.

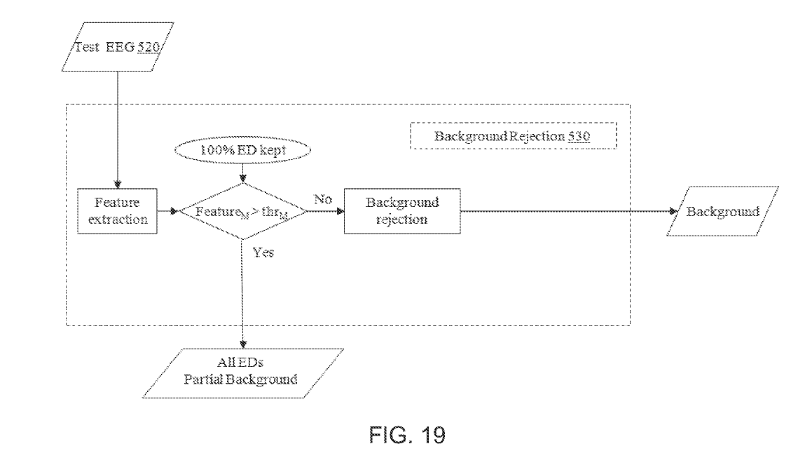

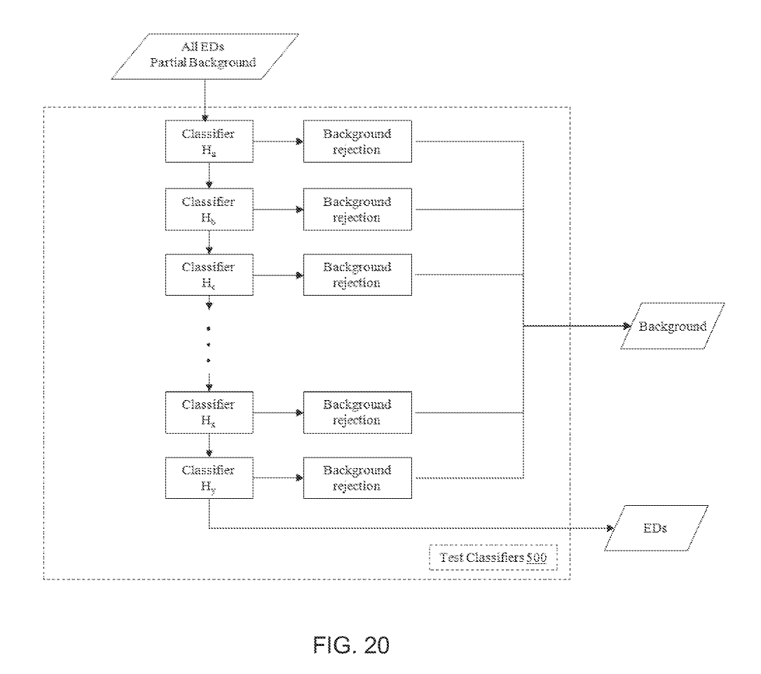

In FIG. 19, simple features (e.g., waveform thresholding, waveform steepness, etc.) are extracted to reject partial backgrounds (e.g., non-EDs). Global cumulative density functions of all the features are derived using all training EEG records for both EDs and backgrounds. The feature with the largest background rejection rate is selected while reserving all or nearly all EDs.

Returning to FIG. 20, the remaining data (all EDs and partial background EEG) after background rejection are input to the trained cascade of classifiers. The background EEG (e.g., non-EDs) is progressively rejected along the queue of classifiers, leaving only EDs at the end.

The steps included in the embodiments illustrated and described in relation to FIGS. 1-20 are not limited to the embodiment shown and may be combined in several different orders and modified within multiple other embodiments. Although disclosed in specific combinations within these figures, the steps disclosed may be independent, arranged and combined in any order and/or dependent on any other steps or combinations of steps.

Other embodiments and uses of the above inventions will be apparent to those having ordinary skill in the art upon consideration of the specification and practice of the invention disclosed herein. The specification and examples given should be considered exemplary only, and it is contemplated that the appended claims will cover any other such embodiments or modifications as fall within the true scope of the invention.

The Abstract accompanying this specification is provided to enable the United States Patent and Trademark Office and the public generally to determine quickly from a cursory inspection the nature and gist of the technical disclosure and in no way intended for defining, determining, or limiting the present invention or any of its embodiments.

The invention claimed is:

1. A system, comprising:
a plurality of electroencephalograph (EEG) electrodes, each EEG electrode being configured to attach to a subject and detect EEG data;
a database configured to store an annotated set of confirmed epileptiform discharges (ED) waveforms;
a computing device coupled to the plurality of EEG electrodes and the database and comprising instructions that, when executed by a processor running on the computing device, cause the computing device to:
receive, from the plurality of EEG electrodes, a signal encoding the EEG data;
generate a user interface displaying a plurality of waveforms based upon at least a portion of the EEG data;
receive, from a user and via the user interface, an initial selection of a portion of one of the plurality of waveforms comprising an ED;
identify, using the initial selection, a list of candidate waveforms including potential EDs by determining an alignment of the initial selection with a portion of one of the plurality of waveforms in the EEG data;
display the list of candidate waveforms on the user interface;
receive, from the user and via the user interface, an identification of a subset of the list of candidate waveforms; and
storing the subset of the list of candidate waveforms as an annotated list of confirmed EDs in the database.

2. The system of claim 1, wherein the computing device is configured to use the identification of the subset of the list of candidate waveforms to train a learning algorithm configured to identify EDs in EEG data.

3. The system of claim 1, wherein the user interface includes a user interface device configured to enable a user to selectively view each waveform in the list of candidate waveforms.

4. The system of claim 1, wherein the list of candidate waveforms is identified by calculating a Euclidean distance (EuD) comprising a sum of the squared distances between at least one data point in the initial selection and a second at least one data point within the EEG data.

5. The system of claim 4, wherein calculating the EuD includes using a triangle inequality to reject any data points in the EEG data outside an accepted region defined about the at least one data point in the initial selection.

6. The system of claim 1, wherein the list of candidate waveforms is generated by aligning the portion of one of the plurality of waveforms in the EEG data with a portion of the initial selection using a DTW algorithm.

7. The system of claim 6, wherein the computing device is configured to filter a waveform in the EEG data from the list of candidate waveforms when a peak-to-trough value for the waveform in the EEG data is less than a threshold.

8. The system of claim 6, wherein the computing device is configured to filter a waveform in the EEG data from the list of candidate waveforms when a portion of the waveform in the EEG data overlaps a portion of one of the candidate waveforms in the list of candidate waveforms.

9. The system of claim 1, wherein prior to generating the list of candidate waveforms, the EEG data is pre-processed by at least one of data compression and data filtering.

10. A system, comprising:
a plurality of electroencephalograph (EEG) electrodes, each EEG electrode being configured to attach to a subject and detect EEG data;
a database storing a plurality of EEG data classifiers, the plurality of EEG data classifiers being trained using an annotated set of confirmed epileptiform discharges (ED) waveforms and EEG background data and being sorted according to specificity;
a computing device comprising instructions that, when executed by a processor running on the computing device, cause the computing device to:
receive, from the plurality of EEG electrodes, EEG data;
filter background data from the EEG data to generate filtered EEG data;
sequentially analyze the filtered EEG data with each one of the plurality of EEG data classifiers to identify a plurality of candidate waveforms including potential EDs; and
generate a user interface displaying the plurality of candidate waveforms.

11. The system of claim 10, wherein the EEG data classifiers include extreme learning machine classifiers, support vector machine classifiers, or support vector regression classifiers.

12. The system of claim 10, wherein background data is removed from the EEG data using a most simple classifier in the plurality of EEG data classifiers.

13. The system of claim 10, wherein the plurality of EEG data classifiers have a sensitivity score of at least 99.9% to potential EDs.

14. The system of claim 10, wherein the set of confirmed ED waveforms are derived from EEG data from a plurality of subjects.

15. A method, comprising the steps of:
storing, in a database, an annotated set of confirmed epileptiform discharges (ED) waveforms
receiving, by a computing device coupled to the database, a signal encoding electroencephalograph (EEG) data from a plurality of electrodes each attached to a subject and detecting EEG data, the plurality of electrodes being coupled to the computing device;
generating, by the computing device, a user interface displaying a plurality of waveforms based upon at least a portion of the EEG data;
receiving, by the computing device, from a user and via the user interface, an initial selection of a portion of one of the plurality of waveforms comprising an ED;
identifying, by the computing device, using the initial selection, a list of candidate waveforms including potential EDs by determining an alignment of the initial selection with a portion of one of the plurality of waveforms in the EEG data;
displaying, by the computing device, the list of candidate waveforms on the user interface;
receiving, by the computing device, from the user and via the user interface, an identification of a subset of the list of candidate waveforms; and
storing, by the computing device, the subset of the list of candidate
waveforms as an annotated list of confirmed EDs in the database.

16. The method of claim 15, wherein the list of candidate waveforms is identified by calculating a Euclidean distance (EuD) comprising a sum of the squared distances between at least one data point in the initial selection and a second at least one data point within the EEG data.

17. The method of claim 15, wherein the list of candidate waveforms is generated by aligning the portion of one of the plurality of waveforms in the EEG data with a portion of the initial selection using a dynamic time warp (DTW) algorithm.

18. The method of claim 17, further comprising the step of filtering, by the computing device, a waveform in the EEG data from the list of candidate waveforms when a peak-to-trough value for the waveform in the EEG data is less than a threshold.

19. The method of claim 17, further comprising the steps of filtering, by the computing device, a waveform in the EEG data from the list of candidate waveforms when a portion of the waveform in the EEG data overlaps a portion of one of the candidate waveforms in the list of candidate waveforms.

20. The method of claim 15, wherein prior to generating the list of candidate waveforms, the EEG data is pre-processed by at least one of data compression and data filtering.
